(19) United States
(12) Patent Application Publication
(10) Pub. No.: US 2023/0277713 A1
(43) Pub. Date: Sep. 7, 2023

(54) VOLATILE MATERIAL DELIVERY DEVICE

(71) Applicant: CTR CONSULTORIA TECNICA E REPRESENTACOES, LDA, SAMORA CORREIA (PT)

(72) Inventor: Gopal Parthasarathy ANANTH, Racine, WI (US)

(73) Assignee: CTR CONSULTORIA TECNICA E REPRESENTACOES, LDA, SAMORA CORREIA (PT)

(21) Appl. No.: 18/011,595

(22) PCT Filed: Jun. 22, 2021
(86) PCT No.: PCT/IB2021/055505

§ 371 Date: Dec. 20, 2022

Related U.S. Application Data
(60) Provisional application No. 63/046,822, filed on Jul. 1, 2020.

(51) Int. Cl. A61L 9/12

(52) U.S. Cl. CPC A61L 9/122

(57) ABSTRACT

A volatile material delivery device may include a hub having one or more vanes coupled to the hub. A porous material may be coupled to the hub. Optionally, the porous material may be coupled directly to the hub and/or may be coupled indirectly to the hub by being coupled to a vane that is coupled to the hub. A volatile material may be in communication with the porous material so that the volatile material may pass through the porous material. Rotation of the hub may result in rotation of the vanes and the porous material. Additionally, rotation of the porous material may cause or increase the release of volatile material, and rotation of the vanes may generate an air current which may move across the porous material so that the volatile material is distributed into the environment in the air current generated by the vanes.

### CROSS REFERENCE TO RELATED APPLICATIONS

This application claims priority to and the benefit of the filing date of U.S. Provisional Application No. 63/046,822, filed on Jul. 1, 2020, entitled “VOLATILE MATERIAL DELIVERY DEVICE”, which is hereby incorporated by reference in its entirety.

### FIELD OF THE INVENTION

This patent specification relates to the field of devices for the dispensing and distributing of volatile materials, such as volatile liquids, into the ambient air. More specifically, this patent specification relates to a low powered device for the controlled delivery of volatile materials.

### BACKGROUND

Commercially available consumer and institutional products that dispense volatile materials are very popular around the globe; examples include air-fresheners using plug-in devices; and in countries where mosquitoes are a major nuisance one could encounter many systems that evaporate insecticidal actives. These volatile materials may include volatile liquids and solids, such as fragrances, insect control actives, disinfectants, and the like.

Unfortunately, these available products suffer from many draw backs. As an example, many available products utilize an active ingredient that is typically either an insect killing/repellent molecule or a fragrance molecule. The vapor pressures of such molecules are such that these don't evaporate sufficiently fast at room temperatures. Even volatilizing the small amounts of insecticide that are typically needed (of the order of a couple of milligrams, far less than a drop, per hour) is a challenge. Therefore, there is almost always a requirement of some additional input of energy, e.g., heat or air flow. Heat is very simple to generate. But generating required heat using electricity is quite energy intensive, thereby requiring power from a wall outlet, or from a large capacity battery for portable devices, which add consumer limitations.

Air flow can also help to volatilize liquids, similar to a damp fabric drying faster when a fan blows air over it. Small electric motors with fan-blades are extremely energy efficient in generating airflow, but the challenge is “how to bring the active in contact with moving air in order to evaporate it?” Off! Clip-Ons has addressed this problem by soaking the active in an absorbent matrix and blowing air through it. The downsides are that the matrix hangs on to the active tight enough that the performance is not uniform over the product's life, and the matrix chokes the airflow making it an inefficient system. Once the active ingredient is evaporated it needs to spread and fill into the space in order to be effective; this typically requires air flows to distribute it around. Heat generates convective air currents; fans generate forced air motion. Normally convective airflow volume is a fraction of that developed by a fan which explains why heat based volatilization is so energy intensive.

Therefore, a need exists for novel devices for the dispensing and distributing of volatile materials, such as volatile liquids, into the ambient air. A further need exists for novel devices for the delivery of volatile materials that are low powered and energy efficient. There is also a need for novel devices for the controlled delivery of volatile materials.

### BRIEF SUMMARY OF THE INVENTION

A volatile material delivery device is provided. In some embodiments, the device may include a hub having one or more vanes coupled to the hub which may be configured to function as a fan. A porous material may be coupled to the hub. Optionally, the porous material may be coupled directly to the hub and/or may be coupled indirectly to the hub by being coupled to a vane that is coupled to the hub. A volatile material may be in communication with the porous material, such as by being impregnated in the porous material and/or in fluid communication with the porous material, so that the volatile material may pass through the porous material. Rotation of the hub may result in rotation of the vanes and the porous material. Additionally, rotation of the porous material may cause or increase the release of volatile material, and rotation of the vanes may generate an air current which may move across the porous material so that the volatile material is distributed into the environment in the air current generated by the vanes.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a perspective view of an example of a volatile material delivery device according to various embodiments described herein.

FIG. 2 illustrates a perspective view of another example of a volatile material delivery device according to various embodiments described herein.

FIG. 3 shows a perspective view of a further example of a volatile material delivery device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

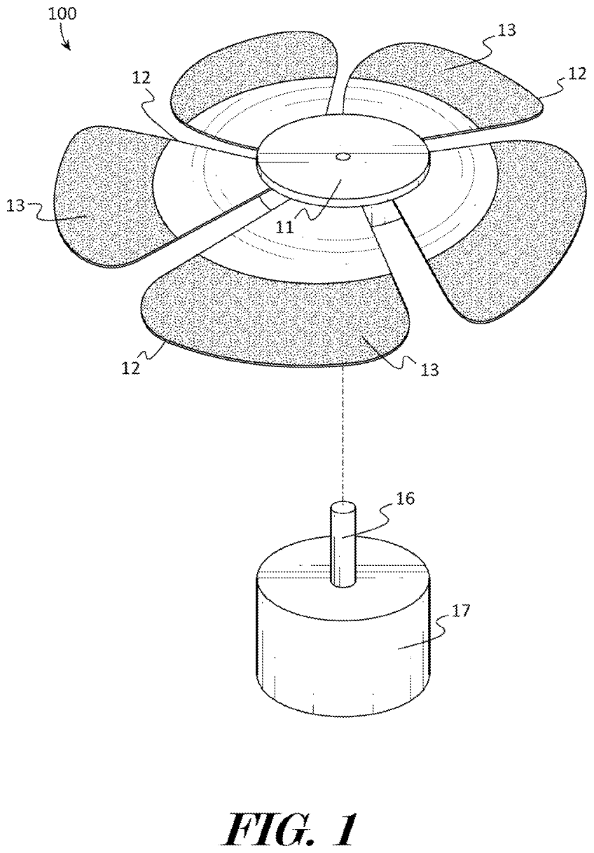

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new low powered device for the controlled delivery of volatile materials is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments. FIG. 1 illustrates an example of a volatile material delivery device (“the device”) 100 according to various embodiments. In some embodiments, the device 100 may comprise a hub 11 and one or more vanes 12 may be coupled to the hub 11. A porous material 13 may be coupled to the hub 11. Optionally, the porous material 13 may be coupled directly to the hub 11. Optionally, the porous material 13 may be coupled indirectly to the hub 11 by being coupled to a vane 12 that is coupled to the hub 11. The porous material 13 may be in communication with a volatile material so that the volatile material may pass through the porous material 13. The hub 11 may be rotated resulting in rotation of the vanes 12 and the porous material 13. Rotation of the porous material 13 may cause or increase the release of volatile material, and rotation of the vanes 12 may generate an air current which may move across the porous material 13 so that the volatile material is distributed into the environment in the air current generated by the vanes 12.

Generally, the device 100 may be configured to provide controlled release of a volatile material into the environment around the device 100. In preferred embodiments, a volatile material may comprise a volatile liquid. Example volatile liquids include liquid fragrances, liquid deodorizers, liquid insect repellents, and liquid disinfectants. In further embodiments, a volatile material may comprise a volatile solid. Example volatile solids include solid fragrances, solid deodorizers, solid insect repellents, and solid disinfectants. It should be understood that a volatile material may comprise any material capable of being volatilized into air.

The device 100 may comprise a hub 11 to which one or more vanes 12 and a porous material 13 may be coupled. Generally, rotation of the hub 11 may cause rotation of the vanes 12 and porous material 13. A hub 11 and one or more vanes 12 may function as or form a fan. A fan is a powered machine used to create flow within a fluid, typically a gas such as air via a rotating arrangement of vanes or blades which act on the air. The rotating assembly of vanes 12 or blades and a hub 11 is known as an impeller, rotor, or runner. The vanes 12 and hub 11 may be configured in any size and shape to form any type of fan. Additionally, the vanes 12 may be shaped and arranged to form any type of structure that is capable of generating air currents when rotated.

In some embodiments, the vanes 12 and hub 11 may be shaped and arranged to form an axial-flow fan. Axial-flow fans have blades that force air to move parallel to the shaft about which the blades rotate. In some embodiments, the vanes 12 and hub 11 may be shaped and arranged to form a centrifugal fan. A centrifugal fan, sometimes called a “scroll fan”, has a moving component (called an impeller) that consists of a central shaft (hub 11) about which a set of blades (vanes 12) that form a spiral, or ribs, are positioned. Centrifugal fans blow air at right angles to the intake of the fan, and spin the air outwards to the outlet (by deflection and centrifugal force). The impeller rotates, causing air to enter the fan near the shaft and move perpendicularly from the shaft to the opening in the scroll-shaped fan casing. In some embodiments, the vanes 12 and hub 11 may be shaped and arranged to form a cross-flow or tangential fan, sometimes known as a tubular fan. This type of fan is usually long in relation to the diameter, so the flow remains approximately two-dimensional away from the ends. The cross-flow fan uses an impeller with forward-curved blades or vanes 12, which may be placed in a housing consisting of a rear wall and a vortex wall. Unlike radial machines, the main flow moves transversely across the impeller, passing the blading twice.

A hub 11 and vanes 12 may be made from any material which is suitable for contacting air and resisting torsion and other stresses that result from rotation. For example, a hub 11 and vanes 12 may be made from or comprise substantially rigid materials, such as metal and metal alloys, hard plastics, including polyethylene (PE), Ultra-high-molecular-weight polyethylene (UHMWPE, UHMW), polypropylene (PP) and polyvinyl chloride (PVC), polycarbonate, nylon, hard rubbers, wood, other plant based materials; cushioning materials, such as silicone foams, rubber foams, urethane foams including plastic foams, neoprene foam, latex foam rubber, polyurethane foam rubber, or elastomer materials such as elastic plastics, elastic silicone, elastic rubbers; and/or any other material including combinations of materials. In some embodiments, a hub 11 and/or one or more vanes 12 may comprise a porous material 13.

The device 100 may comprise a porous material 13 which may be coupled with a hub 11 and/or one or more vanes 12. Generally, a porous material 13 may comprise any material that is permeable to a volatile material. A porous material 13 or a porous medium may comprise a material containing pores or voids, and the pores or voids may allow a volatile material to be communicated through the porous material 13 via the pores or voids. In preferred embodiments, a porous material 13 may be or may comprise a porous plastic or polymer. In some embodiments, a porous material 13 may be or may comprise a sintered porous plastic having various polymers or particles that are fused to create controlled pore sizes, which creates a strong, self-supporting material. In further embodiments, a porous material 13 may be or may comprise bonded polymer fibers. In still further embodiments, a porous material 13 may be or may comprise an open-cell polymer foam. In further embodiments, a porous material 13 may be or may comprise wood, such as cork, ceramics or the like.

The size of the pores or voids and number of the pores or voids in the porous material 13 may govern the rate at which a volatile material may be communicated through the porous material 13. For example, increasing the size of the pores or voids and/or number of the pores or voids in the porous material 13 may increase the rate at which a volatile material may be communicated through the porous material 13, while decreasing the size of the pores or voids and/or number of the pores or voids in the porous material 13 may decrease the rate at which a volatile material may be communicated through the porous material 13.

The device 100 may comprise a porous material 13 which may be coupled with a hub 11 and/or one or more vanes 12 so that rotation of the hub 11 and vanes 12 results in rotation of the porous material 13.

In some embodiments, and as shown in FIG. 1, the device 100 may comprise a porous material 13 which may be coupled to one or more vanes 12 so that rotation of the hub 11 and vanes 12 results in rotation of the porous material 13. In this manner, the porous material 13 may be coupled indirectly to the hub 11. In preferred embodiments, a porous material 13 may be integrally formed with a vane 12 so that the porous material 13 forms part of the vane 12. In further embodiments, a porous material 13 may be coupled to a vane 12, and optionally removably coupled to a vane 12 so as to allow the porous material 13 to be replaced when its volatile material is exhausted. A porous material 13 may form portions of the vane 12 distal to the hub 11, proximate to the hub 11, or any other portion of the vane 12. In further embodiments, a vane 12 may be formed entirely or approximately entirely out of a porous material 13. Preferably, rotation of the vane 12 may exert centrifugal force on a volatile material in the porous material 13 to cause or increase the rate at which the volatile material is released from the porous material 13 into the air current generated by the vane 12 as it travels across the porous material 13.

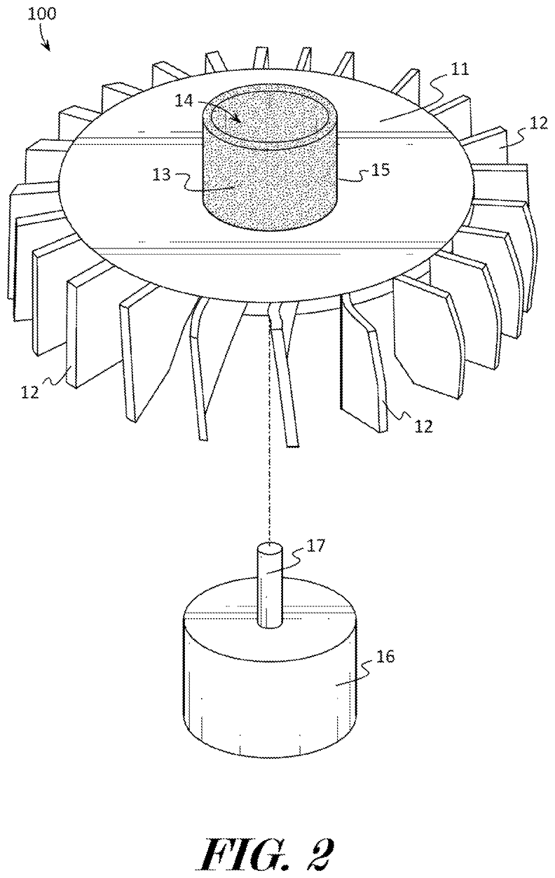

In some embodiments, and as shown in FIG. 2, the device 100 may comprise a porous material 13 which may be coupled to the hub 11 so that rotation of the hub 11 and vanes 12 results in rotation of the porous material 13. In this manner, the porous material 13 may be coupled directly to the hub 11. In further embodiments, a porous material 13 may be coupled to a hub 11, and optionally removably coupled to a hub 11, so as to allow the porous material 13 to be replaced when its volatile material is exhausted. Preferably, rotation of the hub 11 may exert centrifugal force on a volatile material in the porous material 13 to cause or increase the rate at which the volatile material is released from the porous material 13 into the air current generated by the vanes 12 as it travels across the porous material 13.

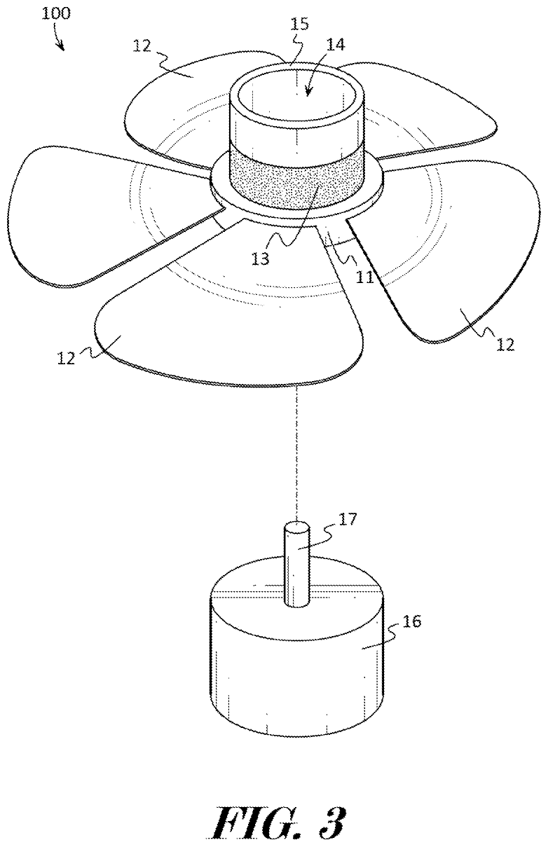

In some embodiments, and as shown in FIG. 3, the device 100 may comprise a reservoir cavity 14 which may be formed by a reservoir 15 that may be coupled to the hub 11. Generally, a reservoir 15 may function as a container for holding a volume of a volatile material within its reservoir cavity 14. In this manner, a user may add a volume of a volatile material to the device 100, such as for refilling a volatile material or changing the volatile material that may be dispensed by the device 100. In some embodiments, the reservoir 15 may be coupled to a porous material 13 so that the reservoir cavity 14 may be in communication with the porous material 13 to enable a volatile material within the reservoir cavity 14 to pass into the porous material 13. In further embodiments, a porous material 13 may form all or portions of a reservoir 15 may be coupled to the hub 11 so that all or portions of the reservoir cavity 14 may be bound or formed with the porous material 13 to enable a volatile material within the reservoir cavity 14 to pass into the porous material 13. Preferably, rotation of the hub 11 may exert centrifugal force on a volatile material in the porous material 13 to cause or increase the rate at which the volatile material is released from the porous material 13 into the air current generated by the vanes 12 as it travels across the porous material 13.

In further embodiments, a porous material 13 may be integrally formed with a hub 11 so that the porous material 13 forms all or parts of the hub 11. In still further embodiments, a hub 11 may be formed entirely or approximately entirely out of a porous material 13.

In some embodiments, the device 100 may comprise a motor 16 which may be operable to cause the rotation of the vanes 12, such as by rotating the hub 11. Optionally, the hub 11 may be coupled to directly to a motor 16, such as to the drive shaft 17 of the motor 16, or coupled indirectly to a motor 16 via a mechanical arrangement which provides controlled application of power, such as a gearbox that uses gears and gear trains to provide speed and torque conversions from a rotating power source to another device.

Generally, a motor 16 may comprise any device which may be used to rotate the hub 11 and vanes 12. In some embodiments, motor 16 may comprise a brushed DC motor, brushless DC motor, switched reluctance motor, universal motor, AC polyphase squirrel-cage or wound-rotor induction motor, AC SCIM split-phase capacitor-start motor, AC SCIM split-phase capacitor-run motor, AC SCIM split-phase auxiliary start winding motor, AC induction shaded-pole motor, wound-rotor synchronous motor, hysteresis motor, synchronous reluctance motor, pancake or axial rotor motor, stepper motor, or any other type of motor. In further embodiments, a motor 16 may comprise a hydraulic motor such as a Gear and vane motor, Gerotor motor, Axial plunger motors, Radial piston motors, or any other hydraulically motivated motor. In still further embodiments, a motor 16 may comprise a pneumatic motor, such as a linear pneumatic motor and a pneumatic rotary vane motor.

In some embodiments, the device 100 may comprise a comprise a power source which may provide electrical power to an electrical motor 16. A power source may comprise a battery, such as a lithium ion battery, nickel cadmium battery, alkaline battery, or any other suitable type of battery, a fuel cell, a capacitor, a super capacitor, or any other type of energy storing and/or electricity releasing device. In further embodiments, a power source may comprise a power cord, kinetic or piezo electric battery charging device, a solar cell or photovoltaic cell, and/or inductive charging or wireless power receiver. In further embodiments, a power source may comprise a power charging and distribution module which may be configured to control the recharging of the power source and discharging of the power source.

While some exemplary shapes and sizes have been provided for elements of the device 100, it should be understood to one of ordinary skill in the art that the hub 11, vanes 12, porous material 13, reservoir 15, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A volatile material delivery device, the device comprising:
a hub having one or more vanes coupled to the hub;
a porous material may be coupled to the hub, wherein the porous material may be coupled directly to the hub and/or may be coupled indirectly to the hub by being coupled to a vane that is coupled to the hub; and
a volatile material may be in communication with the porous material so that the volatile material may pass through the porous material, wherein rotation of the hub results in rotation of the vanes and the porous material, and wherein rotation of the porous material may cause or increase the release of volatile material, and rotation of the vanes may generate an air current which may move across the porous material so that the volatile material is distributed into the environment in the air current generated by the vanes.
